(19) United States
(12) Patent Application Publication
(10) Pub. No.: US 2006/0286662 A1
(43) Pub. Date: Dec. 21, 2006

(54) Medical diagnostic test strip

(76) Inventor: Chien-Chen Chen, Taipei (TW)

Correspondence Address: BRUCE H. TROXELL, SUITE 1404, 5205 LEESBURG PIKE, FALLS CHURCH, VA, 22041, US

(21) Appl. No.: 11/455,863

(22) Filed: Jun. 20, 2006

(30) Foreign Application Priority Data
Jun. 21, 2005 (TW) 094210422

(51) Int. Cl. C12M 1/34

(52) U.S. Cl. 435/287.2; 977/900

(57) ABSTRACT

A medical diagnostic test strip is disclosed, which comprises: a top plate, having at least a cell arranged therein for storing an enzyme, each cell having at least a microchannel channeling out to the outer rim of the top plate therefrom while enabling one of the plural microchannel to guide in an analyte by siphoning; and a bottom plate, capable of being coupled correspondingly to the top plate, having at least a conductive wire, being arranged and extending from a position of the bottom plate corresponding to each cell while keeping each conductive wire from contacting the enzyme.

### FIELD OF THE INVENTION

The present invention relates to a medical diagnostic test strip, and more particularly, to a medical diagnostic test strip capable of keeping its conductive wires from contacting an enzyme for preventing the conductive wires from redox reaction and thus preserving the electrical characteristics of the conductive wires.

### BACKGROUND OF THE INVENTION

Following the advance of medical technology, most diseases can be cured or prevented enabling people, male or female, have a longer life than ever. Nowadays, people can do self-inspection by means of some scientific inspection methods and provide data of inspection for doctors to treat the diseases properly.

Diabetes is one of the modern diseases, which is caused by the pancreas unable to secrete insulin (or insufficient insulin). Insulin decomposes glucose in human blood and transfers it into useful energy. The diabetes patient cannot effectively decompose glucose such that the glucose is accumulated in blood increasing the probability of apoplexy, myocardial infarction, blindness and kidney exhaustion. Therefore, the diabetes patient has to take medicine regularly and go to hospital for follow-up treatment to prevent related complications.

Laboratory tests, such as blood test and urine test, are tools helpful in evaluating the health status of an individual. Any unusual or abnormal results should be discussed with your physician. It is not possible to diagnose or treat any disease or problem with this blood test alone. It can, however, help you to learn more about your body and detect potential problems in early stages.

Therefore, analyte detection in physiological fluids, e.g. blood or blood derived products, is of ever increasing importance to today's society. Analyte detection assays find use in a variety of applications, including clinical laboratory testing, home testing, etc., where the results of such testing play a prominent role in diagnosis and management in a variety of disease conditions. One conventional method that is employed for analyte detection is an electrochemical method. In such methods, an aqueous liquid sample is placed into a reaction zone in an electrochemical cell of a test strip comprising two electrodes, i.e. a reference and working electrode, where the electrodes have an impedance which renders them suitable for amperometric measurement. The component to be analyzed is allowed to react directly with an electrode, or directly or indirectly with a redox reagent like an enzyme to form an oxidizable (or reducible) substance in an amount corresponding to the concentration of the component to be analyzed, i.e. analyte. The quantity of the oxidizable (or reducible) substance present is then estimated electrochemically and related to the amount of analyte present in the initial sample. Nevertheless, the rodex reagent is prone to react with the two electrodes causing the erosion of the same that will eventually cause the test strip to be damaged. Moreover, if an analyzer fails to detect a damaged test strip and keeps on using the test strip for analyte detection, the outcome might be fatal since the redox reaction will cause the impedance of the two electrodes to increase and thus produce a false reading thereby which might eventually cause a false diagnosis.

### SUMMARY OF THE INVENTION

In view of the disadvantages of prior art, the primary object of the present invention is to provide a medical diagnostic test strip capable of keeping its conductive wires from contacting an enzyme for preventing the conductive wires from redox reaction, such that the test strip can be preserved for a comparatively longer time without being damaged.

It is another object of the invention to provide a conductive wire with improved oxidation resistance for the aforesaid medical diagnostic test strip, which is made of a material selected from the group consisting of carbon nano tube (CNT) and metal particles and is further being coated with an epoxy resin compound of oxidation resistant and acid/alkali resistant capability.

Yet, another object of the invention is to provide a plate as a component of the aforesaid medical diagnostic test strip, that the plate has at least a microchannel and at least a cell, all being integrally formed thereon by a simple press molding process utilizing a die.

To achieve the above objects, the present invention provides a medical diagnostic test strip, comprising:
a top plate, having at least a cell arranged therein for storing for storing one of a plurality of enzymes, each cell having at least a microchannel channeling out to the outer rim of the top plate therefrom while enabling one of the plural microchannel to guide in an analyte by siphoning; and
a bottom plate, capable of being coupled correspondingly to the top plate, having at least a conductive wire, being arranged at and extending from a position of the bottom plate corresponding to each cell of the top plate while keeping each conductive wire from contacting the enzyme.

In a preferred aspect, the present invention provides a medical diagnostic test strip, comprising:
a top plate, having at least a cell arranged therein for storing one of a plurality of enzymes, each cell having at least a microchannel channeling out to the outer rim of the top plate therefrom while enabling one of the plural microchannel to guide in an analyte by siphoning;
a bottom plate, having a cell arranged therein for storing an enzyme, the cell having at least two microchannels channeling out to the outer rim of the bottom plate therefrom while enabling one of the plural microchannel to guide in the analyte by siphoning; and
a middle plate, capable of being coupled correspondingly to the top plate and the bottom in respective, having at least a conductive wire, being arranged and extending from a position of the middle plate corresponding to each cell of the top plate while keeping each conductive wire from contacting the enzyme thereof, and at least a conductive wire, being arranged and extending from a position of the middle plate corresponding to each cell of the bottom plate while keeping each conductive wire from contacting the enzyme thereof.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1A is an exploded view of a two-piece medical diagnostic test strip according to a first embodiment of the invention.

FIG. 1B shows respectively a schematic diagram of the top plate of FIG. 1A having a plurality of microchannels and a cell arranged thereon, and a schematic diagram of the bottom plate of FIG. 1A having extending conductive wires arranged thereon.

FIG. 1C shows respectively an A-A cross-section and B-B cross-section of the top plate shown in FIG. 1B.

FIG. 1D is an exploded view of a two-piece medical diagnostic test strip according to a second embodiment of the invention.

FIG. 1E is an exploded view of a two-piece medical diagnostic test strip according to a third embodiment of the invention.

FIG. 1F is a schematic diagram showing a microchannel having a breach being arranged at the outer rim of the top plate according to the present invention.

FIG. 2A is exploded view of a three-piece medical diagnostic test strip according to a fourth embodiment of the invention.

FIG. 2B is exploded view of a three-piece medical diagnostic test strip according to a five embodiment of the invention.

FIG. 2C is exploded view of a three-piece medical diagnostic test strip according to a sixth embodiment of the invention.

### DESCRIPTION OF THE PREFERRED EMBODIMENT

For your esteemed members of reviewing committee to further understand and recognize the fulfilled functions and structural characteristics of the invention, several preferable embodiments cooperating with detailed description are presented as the follows.

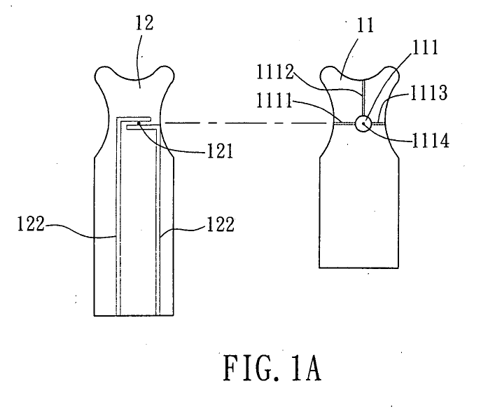

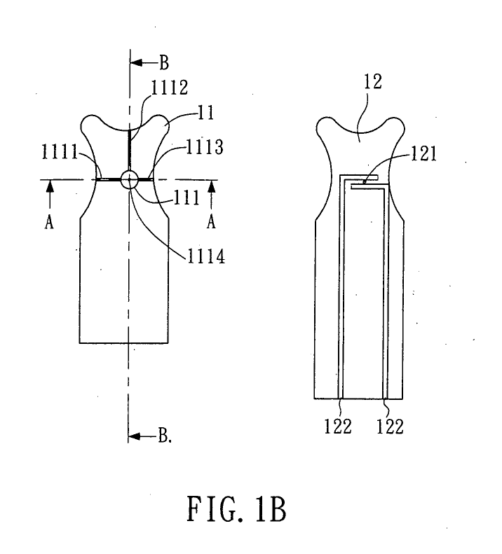

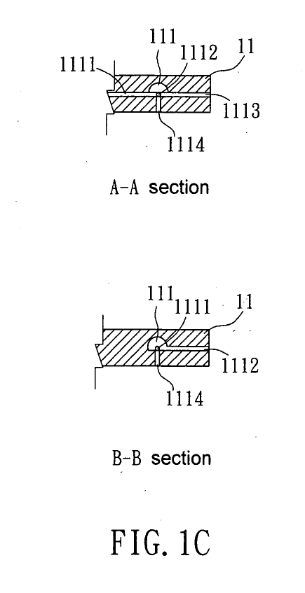

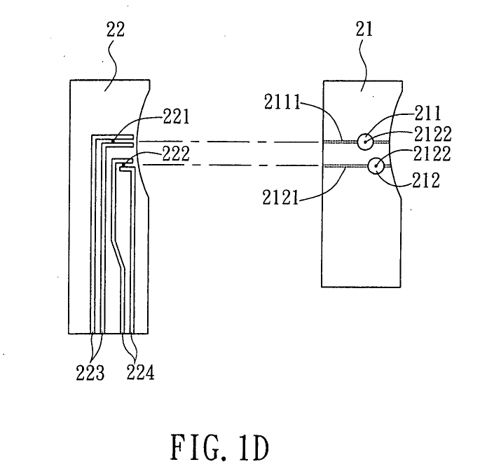

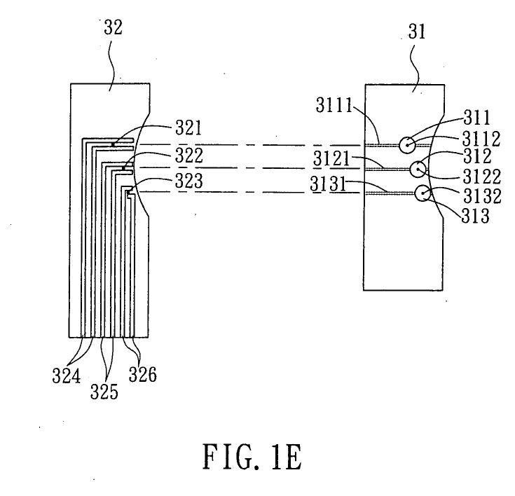

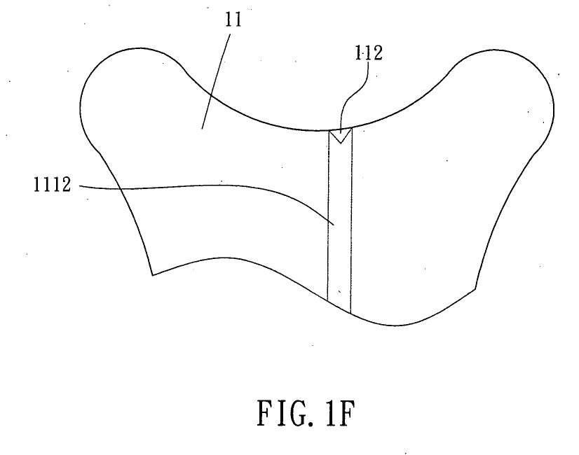

Please refer to FIG. 1A, FIG. 1B, FIG. 1C and FIG. 1D, which are schematic diagrams illustrating a two-piece medical diagnostic test strip according to a first embodiment of the invention. The two-piece medical diagnostic test strip, being adapted to a biomonitor, comprises:
a top plate 11, having at least a cell 111 arranged therein for storing one of a plurality of enzymes (as the one cell shown in FIG. 1A, the two cells shown in FIG. 1D and the three cells shown in FIG. 1 E, however, the number of the cells are not limited by the above embodiments and can be as many as the space of the top plate permitted), each cell 111 having three microchannels 1111, 1112, 1113 channeling out to the outer rim of the top plate therefrom while enabling one of the plural microchannel to guide in an analyte by siphoning; and
a bottom plate 12, capable of being coupled correspondingly to the top plate 11, having at least a conductive wire 122, being arranged at and extending from a position of the bottom plate 12 corresponding to each cell 111 of the top plate 11 while keeping each conductive wire 122 from contacting the enzyme;
wherein, the plural enzymes are being arranged in their corresponding cells respectively during the manufacturing of the top plate; each microchannel and each cell of the top plate 11 are all being integrally formed thereon by a simple press molding process utilizing a die; each microchannel, such as the microchannel 1112, can have a breach 112 arranged at a position thereof in the vicinity of the outer rim of the top plate 11 for breaking the surface tension of the liquid-state analyte and thus helping the guiding of the analyte into each cell 111; at least a side of the top plate has at least one conductive wire arranged thereon for adapting the top plate 11 to the biomonitor while at least a side of the bottom plate 12 has at least one conductive wire 122 arranged thereon for adapting the bottom plate 12 to the biomonitor so as to enable a plurality of medical test to be performed at the same time by using only one test strip; the conductive wire is made of a material selected from the group consisting of carbon nano tube (CNT) and metal particles.

Furthermore, each cell 111 has a first via hole 1114 arranged at the bottom thereof and each first via hole 1114 is connected to a second via hole 121 arranged at a position of the bottom plate 12 corresponding therewith, thereby a conduit is formed for guiding the mixture of the analyte and the corresponding enzyme from the top plate 11 to the bottom plate 12, such that each conductive wire 1122 of the bottom plate 12 will get into contact with the mixture of the analyte and the enzyme after it is being guided thereto through the conduit formed by the connection of the first via hole 1114 and the second via hole 121 and thus a conduction loop can be formed since most of the analyte, such as blood or urine, are conductive. It is noted that the number of the microchannels are not limited by the embodiments shown hereinbefore and thus can be of any numbers.

The minute current generated by the reaction of the enzyme to the analyte can be converted into a voltage by a circuit and then the voltage is detected and valued by the biomonitor so as to generate a report for the testing, that the aforesaid process are fully disclosed in prior arts and thus are not going to described further hereinafter.

Please refer to FIG. 1D, which is an exploded view of a two-piece medical diagnostic test strip according to a second embodiment of the invention. The top plate 21 of the second embodiment has two cells 211, 212 arranged thereon while correspondingly there are two sets of conductive wires 223, 224 arranged on the bottom plate 22. The difference between the test strip shown in FIG. 1A to FIG. 1C and that of FIG. 1D is that: by the disposition of the plural sets of conductive wires, a various of medical tests can be performed at the same time while an analyte is being guided into the plural cells of the test strip, that is, a plurality of medical tests can be performed at the same time using only one test strip of the invention.

Please refer to FIG. 1E, which is an exploded view of a two-piece medical diagnostic test strip according to a third embodiment of the invention. The top plate 31 of the second embodiment has two cells 311, 312, 313 arranged thereon while correspondingly there are three sets of conductive wires arranged on the bottom plate 32. The difference between the test strip shown in FIG. 1A to FIG. 1C and that of FIG. 1E is that: by the disposition of the plural sets of conductive wires, a various of medical tests can be performed at the same time while an analyte is being guided into the plural cells of the test strip, that is, a plurality of medical tests can be performed at the same time using only one test strip of the invention.

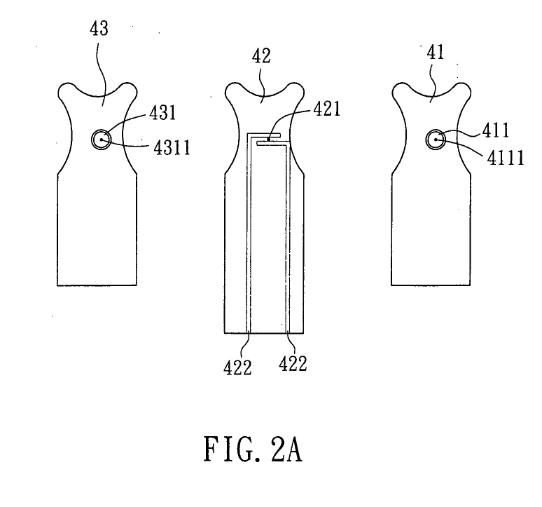

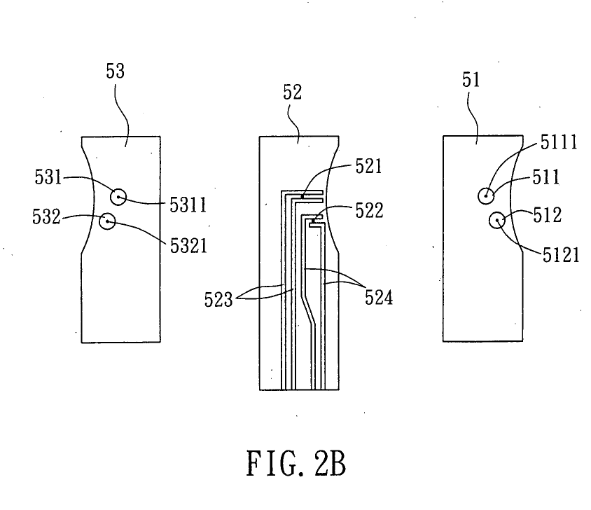

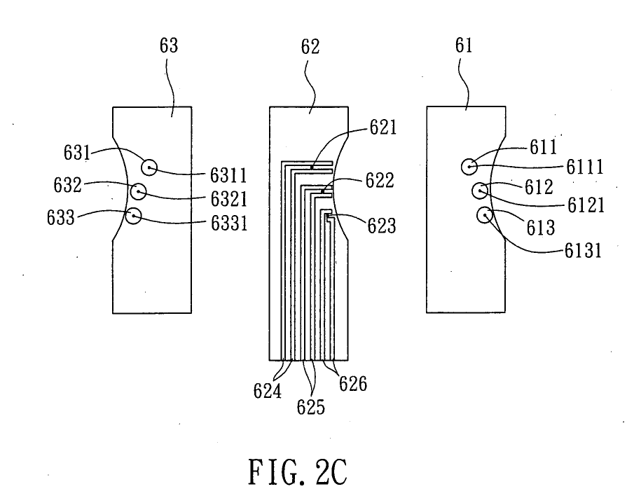

Please refer to FIG. 2A, FIG. 2B and FIG. 2C, which are embodiments of a three-piece medical diagnostic test strip. The three-piece test strip is consisted of a top plate, a middle plate, and a bottom plate; wherein the top plate and the bottom plate have the same configuration while the three plates are formed respectively by a substrate selected from the group consisting of paper and plastic. The three-piece test strip is featured with all the characteristics of the two-piece test strip. However, with the addition of the middle plate, there can be more space in the three-piece test strip available for laying out more microchannels and conductive wires, such that the three-piece test strip may be used to perform more medical tests than that of the two-piece test strip.

From the above description, it is noted that the present invention can provide a medical diagnostic test strip capable of keeping its conductive wires from contacting an enzyme for preventing the conductive wires from redox reaction. Moreover, each conductive wire is made of a material selected from the group consisting of carbon nano tube (CNT) and metal particles and is further being coated with an epoxy resin compound of oxidation resistant and acid/alkali resistant capability, such that the test strip can be preserved for a comparatively longer time without being damaged.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. A medical diagnostic test strip, comprising:
a top plate, having at least a cell arranged therein for storing one of a plurality of enzymes, each cell having at least a microchannel channeling out to the outer rim of the top plate therefrom while enabling one of the plural microchannel to guide in an analyte by siphoning; and
a bottom plate, capable of being coupled correspondingly to the top plate, having at least a conductive wire, being arranged at and extending from a position of the bottom plate corresponding to each cell of the top plate while keeping each conductive wire from contacting the enzyme.

2. The test strip of claim 1, wherein the plural enzymes are being arranged in their corresponding cells respectively during the manufacturing of the top plate.

3. The test strip of claim 1, wherein each cell has a first via hole arranged at the bottom thereof and each first via hole is connected to a second via hole arranged at a position of the bottom plate corresponding therewith, thereby a conduit is formed for guiding the mixture of the analyte and the corresponding enzyme from the top plate to the bottom plate.

4. The test strip of claim 1, wherein at least a side of the top plate has at least one conductive wire arranged thereon.

5. The test strip of claim 1, wherein at least a side of the bottom plate has at least one conductive wire arranged thereon.

6. The test strip of claim 1, wherein the conductive wire is made of a material selected from the group consisting of carbon nano tube (CNT) and metal particles.

7. The test strip of claim 1, wherein the top plate and the bottom plate are formed respectively by a substrate selected from the group consisting of paper and plastic.

8. The test strip of claim 1, wherein the top plate has at least a microchannel and at least a cell, all being integrally formed thereon by a simple press molding process utilizing a die.

9. The test strip of claim 1, wherein a breach is arranged on each microchannel at a position thereof in the vicinity of the outer rim of the top plate for breaking the surface tension of the liquid-state analyte and thus helping the guiding of the analyte into each cell.

10. The test strip of claim 8, wherein a breach is arranged on each microchannel at a position thereof in the vicinity of the outer rim of the top plate for breaking the surface tension of the liquid-state analyte and thus helping the guiding of the analyte into each cell.

11. A medical diagnostic test strip, comprising:
a top plate, having at least a cell arranged therein for storing one of a plurality of enzymes, each cell having at least a microchannel channeling out to the outer rim of the top plate therefrom while enabling one of the plural microchannel to guide in an analyte by siphoning;
a bottom plate, having a cell arranged therein for storing an enzyme, the cell having at least two microchannels channeling out to the outer rim of the bottom plate therefrom while enabling one of the plural microchannel to guide in the analyte by siphoning; and
a middle plate, capable of being coupled correspondingly to the top plate and the bottom in respective, having at least a conductive wire, being arranged and extending from a position of the middle plate corresponding to each cell of the top plate while keeping each conductive wire from contacting the enzyme thereof, and at least a conductive wire, being arranged and extending from a position of the middle plate corresponding to each cell of the bottom plate while keeping each conductive wire from contacting the enzyme thereof.

12. The test strip of claim 7, wherein the plural enzymes are being arranged in their corresponding cells respectively during the manufacturing of the top plate.

13. The test strip of claim 11, wherein each cell has a first via hole arranged at the bottom thereof and each first via hole is connected to a second via hole arranged at a position of the bottom plate corresponding therewith, thereby a conduit is formed for guiding the mixture of the analyte and the corresponding enzyme from the top plate to the bottom plate.

14. The test strip of claim 11, wherein at least a side of the top plate has at least one conductive wire arranged thereon.

15. The test strip of claim 11, wherein at least a side of the bottom plate has at least one conductive wire arranged thereon.

16. The test strip of claim 11, wherein the conductive wire is made of a material selected from the group consisting of carbon nano tube (CNT) and metal particles.

17. The test strip of claim 11, wherein the top plate, the middle plate and the bottom plate are formed respectively by a substrate selected from the group consisting of paper and plastic.

18. The test strip of claim 11, wherein the top plate has at least a microchannel and at least a cell, all being integrally formed thereon by a simple press molding process utilizing a die, while the bottom plate has at least a microchannel and at least a cell, all being integrally formed thereon by a simple press molding process utilizing a die.

19. The test strip of claim 11, wherein a breach is arranged on each microchannel at a position thereof in the vicinity of the outer rim of the top plate for breaking the surface tension of the liquid-state analyte and thus helping the guiding of the analyte into each cell.

20. The test strip of claim 8, wherein a breach is arranged on each microchannel at a position thereof in the vicinity of the outer rim of the top plate for breaking the surface tension of the liquid-state analyte and thus helping the guiding of the analyte into each cell.
